(19) United States
(12) Patent Application Publication
(10) Pub. No.: US 2024/0209370 A1
(43) Pub. Date: Jun. 27, 2024

(54) METHODS OF MODULATING ALK

(71) Applicant: SOCIETE DES PRODUITS NESTLE S.A., Vevey (CH)

(72) Inventors: Nele Gheldof, Cugy (CH); Armand Valsesia, Chavannes-pres-Renens (CH); Jorg Hager, Houtaud (FR)

(21) Appl. No.: 18/522,766

(22) Filed: Nov. 29, 2023

Related U.S. Application Data
(62) Division of application No. 16/764,187, filed on May 14, 2020, now abandoned.

(30) Foreign Application Priority Data
Nov. 21, 2017 (EP) 17202802.9

(51) Int. Cl. C12N 15/113; C12Q 1/6876; G01N 33/50

(52) U.S. Cl. CPC C12N 15/1135; C12Q 1/6876; G01N 33/5023; C12N 2310/11; C12N 2310/14; C12N 2310/141; G01N 2333/912

(57) ABSTRACT

An agent capable of decreasing the activity of ALK for use in supporting weight maintenance and/or treating or preventing obesity.

### CROSS REFERENCE TO RELATED APPLICATIONS

The present application is a divisional of application Ser. No. 16/764,187 filed May 14, 2020, which is a National Stage of International Application No. PCT/EP2018/079923 filed Nov. 1, 2018, which claims priority to European Patent Application No. 17202802.9 filed Nov. 21, 2017, the entire contents of which are incorporated herein by reference.

### SEQUENCE LISTING

The instant application contains a Sequence Listing which has been submitted electronically in XML file format and is hereby incorporated by reference in its entirety. Said XML copy, created on Feb. 23, 2024, is named 3712036_04676_SL.xml and is 15,397 bytes in size.

### FIELD OF THE INVENTION

The present invention relates to agents which are capable of modulating the activity of ALK and the use of such agents in therapy, in particular in supporting weight maintenance and treating obesity. The invention also relates to methods of identifying such agents.

### BACKGROUND TO THE INVENTION

Obesity is a chronic metabolic disorder that has reached epidemic proportions in many areas of the world. Obesity is the major risk factor for serious co-morbidities such as type 2 diabetes mellitus, cardiovascular disease, dyslipidemia and certain types of cancer (World Health Organ. Tech. Rep. Ser. (2000) 894: i-xii, 1-253).

Obesity refers to a condition in which an individual weighs more than usual as a result of excessive accumulation of energy from carbohydrate, fat and the like. The additional weight is typically retained in the form of fat under the skin or around the viscera. Empirical data suggests that a weight loss of at least 10% of the initial weight results in a considerable decrease in the risk of obesity related co-morbidities (World Health Organ. Tech. Rep. Ser. (2000) 894: i-xii, 1-253). However, the capacity to lose weight shows large inter-subject variability.

Obesity is induced when the amount of energy intake exceeds the amount of energy consumed. Thus, in order to ameliorate obesity, a method of decreasing the amount of energy intake from fat, carbohydrate and the like or a method of increasing the amount of energy consumption by promoting in vivo metabolism is desired. Accordingly, improvements in dietary habit and exercise are considered to be effective methods for the prevention and amelioration of obesity and obesity-related disorders.

Although a number of methods are known for promoting weight loss, subjects face the risk of regaining lost weight once a period of weight loss intervention has been completed. Such regression risks reducing or potentially completely reversing any benefits that were associated with the loss of weight.

Accordingly, there remains a significant need not only for improved methods of promoting weight loss, but also for methods for supporting weight maintenance (preventing or reducing the regain of lost weight, and hence supporting maintenance of weight at a level similar to that achieved following weight loss intervention). Such improvements would provide more complete treatments for obesity, thus decreasing the risk of obesity-related disorders.

Obesity is associated with a number of physiological changes in the body including differences in the levels of certain gene products, which are either higher or lower in obese subjects than in individuals with a normal body weight (Singla, Bardoloi and Parkash, World J Diabetes (2010)). Moreover, it has been shown that the blood levels for many of these gene products changes dramatically during a weight loss intervention (Van Dijk et al. Plos One (2010), Viguerie et al. Plos Genetics (2012), Armenise et al. AJCN (2017)).

Most studies have focused on studying genes in obese populations, and how these genes change during weight loss and maintenance programs, but another way of considering potential treatments is to find genes that might protect some people from weight gain. Indeed, certain people, called Constitutional Thin (CT) individuals, have extreme low body mass index (BMI) throughout life despite their normal feeding, exercise and psychological profile (BMI defined in general as lower than 18 kg/m2).

By using large-scale genetic datasets and clinical databases from population-wide biobanks, one can identify genetic variants that associate with the CT phenotype. A possible causality for changes in gene expression around those genetic variants can then be studied by altering the levels of these genes in knockdown experiments in animal models and evaluating the metabolic effect. Using modem molecular biology techniques these can be carried out and repeated on several animals. When such knock-down leads to non-lethal phenotypes, the effect of the reduced gene expression can be assessed using physiological, morphological, molecular and metabolic and readouts such as weight and fat change. Identifying the genes responsible for this resistance to weight gain could lead to new treatments for obesity.

### SUMMARY OF THE INVENTION

The inventors identified a genetic marker located within the ALK (Anaplastic Lymphoma Receptor Tyrosine Kinase) locus in the Constitutional Thin phenotype using genetic and clinical data from the Estonian EGCUT biobank.

To identify whether the ALK gene is involved in the resistance to weight gain of the CT individuals, whole-body RNAi knockdown of the ALK ortholog in Drosophila melanogaster was carried out. This knockdown led to a viable strain with significantly reduced fat accumulation (as measured by triglycerides levels) compared to wild-type.

Accordingly, in one aspect the invention provides an agent capable of decreasing the activity of ALK for use in supporting weight maintenance and/or treating or preventing obesity.

In another aspect, the invention provides the use of an agent capable of decreasing the activity of ALK for supporting weight maintenance.

In another aspect, the invention provides the use of an agent capable of decreasing the activity of ALK for increasing fat-free mass or increasing the ratio fat-free mass to fat mass.

In another aspect, the invention provides the use of an agent capable of decreasing triglyceride levels.

In another aspect, the invention provides the use of an agent capable of decreasing the activity of ALK for improving dyslipidemia.

In another aspect, the invention provides the use of an agent capable of reducing risk of cardiovascular disease (CVD).

In another aspect, the invention provides a method of supporting weight maintenance comprising administering an agent of the invention to a subject in need thereof. In another aspect, the invention provides a method of reducing fat deposition in a subject comprising administering an agent of the invention to a subject in need thereof. In another aspect, the invention provides a method of treating or preventing obesity comprising administering an agent of the invention to a subject in need thereof.

The activity of ALK may be decreased in comparison with the activity in the absence of the agent of the invention. The activity of ALK may be decreased by, for example, at least 1%, 2%, 3%, 4%, 5%, 6%, 7%, 8%, 9%, 10%, 15%, 20%, 25%, 50%, or 75%.

The agent may be, for example, a ALK antagonist or inhibitor, or the agent may decrease the level of ALK in a cell.

In one embodiment, the agent is administered to a subject during or after a weight loss intervention. In a preferred embodiment, the agent is administered to a subject during a weight loss intervention.

The weight loss intervention may be, for example, a diet regimen (e.g. a low-calorie diet) and/or an exercise regimen.

In one embodiment, the agent decreases the level of ALK in a subject. In this context, “level” refers to the amount of ALK and may be measured, for example, by analysing the amount of protein expressed and/or by analysing the amount of the corresponding mRNA present. Preferably, the agent decreases the expression of ALK. For example, siRNAs, shRNAs, miRNAs or antisense RNAs may reduce expression of ALK.

The level of ALK may be decreased in comparison with the level in the absence of the agent of the invention. The level of ALK may be decreased by, for example, at least 1%, 2%, 3%, 4%, 5%, 6%, 7%, 8%, 9%, 10%, 15%, 20%, 25%, 50%, 75% or 100%.

In one embodiment, the agent is selected from the agents listed in Table 1.

In a preferred embodiment, the agent is selected from one or more of acetylcysteine, emodin, and heparin.

In another preferred embodiment, the agent is selected from the group consisting of an siRNA, shRNA, miRNA, antisense RNA, polynucleotide, polypeptide or small molecule. The polypeptide may be, for example, an antibody. Thus, the agent of the invention may be in the form of a polynucleotide encoding an siRNA, shRNA, miRNA or antisense RNA that targets ALK, or a polypeptide (e.g. an antibody). The polynucleotide may be in the form of a vector, such as a viral vector.

The agent of the invention may be an agent identified by a method of the invention.

In another aspect, the invention provides a method of identifying an agent capable of supporting weight maintenance and/or treating or preventing obesity in a subject comprising the steps:
(a) contacting a preparation comprising a ALK polypeptide or polynucleotide with a candidate agent; and
(b) detecting whether the candidate agent affects the activity of the ALK polypeptide or polynucleotide.

The effect on activity of the ALK polypeptide or polynucleotide may be analysed by comparing the activities of the ALK polypeptide or polynucleotide in the presence and absence (i.e. a control experiment) of the candidate agent.

In another aspect, the invention provides a method of identifying an agent that decreases the activity of ALK comprising the steps:
(a) contacting a preparation comprising a ALK polypeptide or polynucleotide with a candidate agent; and
(b) detecting whether the candidate agent affects the activity of the ALK polypeptide or polynucleotide.

The methods of the invention may be methods for identifying an agent capable of suppressing the appetite of a subject, increasing or prolonging satiety, reducing food intake by a subject and/or reducing fat deposition in a subject.

In one embodiment, the preparation comprising the ALK polypeptide or polynucleotide comprises a cell comprising the ALK polypeptide or polynucleotide.

In a preferred embodiment, the cell is an adipocyte.

In a preferred embodiment, the cell is a brain cell.

In one embodiment, the method is for identifying an agent that decreases the expression of ALK.

In one embodiment, the candidate agent is a natural product, preferably a compound naturally occurring in plants.

In another aspect, the invention provides the use of ALK, or a polynucleotide encoding the same, in a method of identifying an agent that supports weight maintenance, suppresses the appetite of a subject, increases or prolongs satiety, reduces food intake by a subject, reduces fat deposition in a subject, and/or treats or prevents obesity.

In another aspect, the invention provides the use of an agent capable of decreasing the activity of ALK for manufacturing a medicament for use in supporting weight maintenance, suppressing the appetite of a subject, increasing or prolonging satiety, reducing food intake by a subject, reducing fat deposition in a subject, and/or treating or preventing obesity.

In another aspect, the invention provides a method of identifying an agent that decreases the expression of ALK comprising the steps:
(a) contacting a cell, preferably a cell expressing the ALK, with a candidate agent; and
(b) detecting whether the candidate agent decreases the expression of the ALK.

### DESCRIPTION OF THE DRAWINGS

FIG. 1

A Manhattan plot identifying the region of the ALK gene on chromosome 2.Variant position is indicated on the X-axis, statistical significance (−log 10 p-value) is indicated on the Y axis. Each point corresponds to a variant. Gene positions are indicated in the lower panel. The peak lines in the upper panel (secondary Y-axis on the right) indicate recombination rates (in centimorgans per megabase). The top associated variant is indicated with a diamond shape and its chromosome coordinate.

FIG. 2

Whole body RNAi knockdown of Alk reduces triglyceride levels and increases body weight in Drosophila. (A) Triglyceride levels are decreased in Alk RNAi flies. Body weight increased in Alk RNAi flies. (B) Data are represented as mean values. The white bars show data for the wild-type fly (Actin-Gal4/+) and the black bars show data for the RNAi knockdown fly (Actin-Gal4>UAS-AlkIR)*=Z-score lower or higher than −1.95 or 1.95 respectively.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

### ALK

This gene encodes a receptor tyrosine kinase, which belongs to the insulin receptor superfamily. This protein comprises an extracellular domain, a hydrophobic stretch corresponding to a single pass transmembrane region, and an intracellular kinase domain. It plays an important role in the development of the brain and exerts its effects on specific neurons in the nervous system. This gene has been found to be rearranged, mutated, or amplified in a series of tumours including anaplastic large cell lymphomas, neuroblastoma, and non-small cell lung cancer. The chromosomal rearrangements are the most common genetic alterations in this gene, which result in creation of multiple fusion genes in tumourigenesis. Mice homozygous for a null allele show increased ethanol consumption and increased sedation in response to ethanol. Male mice homozygous for a different null allele show delayed puberty, hypogonadotropic hypogonadism, reduced serum testosterone levels, and altered seminiferous tubule morphology.

In Human, the ALK gene is also known as CD246; NBLST3.

In one embodiment, the ALK is human ALK.

An example amino acid sequence of the ALK is the sequence deposited under NCBI Accession No. NP_004295.2 (ALK tyrosine kinase receptor isoform 1 precursor). This variant (1) encodes the longer isoform.

An example amino acid sequence of the ALK is:

(SEQ ID NO: 1)
MGAIGLLWLLPLLLSTAAVGSGMGTGQRAGSPAAGPPLQPREPLSYSRLQ
RKSLAVDFVVPSLFRVYARDLLLPPSSSELKAGRPEARGSLALDCAPLLR
LLGPAPGVSWTAGSPAPAEARTLSRVLKGGSVRKLRRAKQLVLELGEEAI
LEGCVGPPGEAAVGLLQFNLSELFSWWIRQGEGRLRIRLMPEKKASEVGR
EGRLSAAIRASQPRLLFQIFGTGHSSLESPTNMPSPSPDYFTWNLTWIMK
DSFPFLSHRSRYGLECSFDFPCELEYSPPLHDLRNQSWSWRRIPSEEASQ
MDLLDGPGAERSKEMPRGSFLLLNTSADSKHTILSPWMRSSSEHCTLAVS
VHRHLQPSGRYIAQLLPHNEAAREILLMPTPGKHGWTVLQGRIGRPDNPF
RVALEYISSGNRSLSAVDFFALKNCSEGTSPGSKMALQSSFTCWNGTVLQ
LGQACDFHQDCAQGEDESQMCRKLPVGFYCNFEDGFCGWTQGTLSPHTPQ
WQVRTLKDARFQDHQDHALLLSTTDVPASESATVTSATFPAPIKSSPCEL
RMSWLIRGVLRGNVSLVLVENKTGKEQGRMVWHVAAYEGLSLWQWMVLPL
LDVSDRFWLQMVAWWGQGSRAIVAFDNISISLDCYLTISGEDKILQNTAP
KSRNLFERNPNKELKPGENSPRQTPIFDPTVHWLFTTCGASGPHGPTQAQ
CNNAYQNSNLSVEVGSEGPLKGIQIWKVPATDTYSISGYGAAGGKGGKNT
MMRSHGVSVLGIFNLEKDDMLYILVGQQGEDACPSTNQLIQKVCIGENNV
IEEEIRVNRSVHEWAGGGGGGGGATYVFKMKDGVPVPLIIAAGGGGRAYG
AKTDTFHPERLENNSSVLGLNGNSGAAGGGGGWNDNTSLLWAGKSLQEGA
TGGHSCPQAMKKWGWETRGGFGGGGGGCSSGGGGGGYIGGNAASNNDPEM
DGEDGVSFISPLGILYTPALKVMEGHGEVNIKHYLNCSHCEVDECHMDPE
SHKVICFCDHGTVLAEDGVSCIVSPTPEPHLPLSLILSVVTSALVAALVL
AFSGIMIVYRRKHQELQAMQMELQSPEYKLSKLRTSTIMTDYNPNYCFAG
KTSSISDLKEVPRKNITLIRGLGHGAFGEVYEGQVSGMPNDPSPLQVAVK
TLPEVCSEQDELDFLMEALIISKFNHQNIVRCIGVSLQSLPRFILLELMA
GGDLKSFLRETRPRPSQPSSLAMLDLLHVARDIACGCQYLEENHFIHRDI
AARNCLLTCPGPGRVAKIGDFGMARDIYRASYYRKGGCAMLPVKWMPPEA
FMEGIFTSKTDTWSFGVLLWEIFSLGYMPYPSKSNQEVLEFVTSGGRMDP
PKNCPGPVYRIMTQCWQHQPEDRPNFAIILERIEYCTQDPDVINTALPIE
YGPLVEEEEKVPVRPKDPEGVPPLLVSQQAKREEERSPAAPPPLPTTSSG
KAAKKPTAAEISVRVPRGPAVEGGHVNMAFSQSNPPSELHKVHGSRNKPT
SLWNPTYGSWFTEKPTKKNNPIAKKEPHDRGNLGLEGSCTVPPNVATGRL
PGASLLLEPSSLTANMKEVPLFRLRHFPCGNVNYGYQQQGLPLEAATAPG
AGHYEDTILKSKNSMNQPGP

An example nucleotide sequence encoding the ALK is the sequence deposited under NCBI Accession No. NM 004304.4

An example nucleotide sequence encoding the ALK is:

(SEQ ID NO: 2)
AGCTGCAAGTGGCGGGCGCCCAGGCAGATGCGATCCAGCGGCTCTGGGGGCGGCAG
CGGTGGTAGCAGCTGGTACCTCCCGCCGCCTCTGTTCGGAGGGTCGCGGGGCACCGA
GGTGCTTTCCGGCCGCCCTCTGGTCGGCCACCCAAAGCCGCGGGCGCTGATGATGGG
TGAGGAGGGGGCGGCAAGATTTCGGGCGCCCCTGCCCTGAACGCCCTCAGCTGCTGC
CGCCGGGGCCGCTCCAGTGCCTGCGAACTCTGAGGAGCCGAGGCGCCGGTGAGAGC
AAGGACGCTGCAAACTTGCGCAGCGCGGGGGCTGGGATTCACGCCCAGAAGTTCAGC
AGGCAGACAGTCCGAAGCCTTCCCGCAGCGGAGAGATAGCTTGAGGGTGCGCAAGAC
GGCAGCCTCCGCCCTCGGTTCCCGCCCAGACCGGGCAGAAGAGCTTGGAGGAGCCAA
AAGGAACGCAAAAGGCGGCCAGGACAGCGTGCAGCAGCTGGGAGCCGCCGTTCTCAG
CCTTAAAAGTTGCAGAGATTGGAGGCTGCCCCGAGAGGGGACAGACCCCAGCTCCGA
CTGCGGGGGGCAGGAGAGGACGGTACCCAACTGCCACCTCCCTTCAACCATAGTAGTT
CCTCTGTACCGAGCGCAGCGAGCTACAGACGGGGGCGCGGCACTCGGCGCGGAGAG
CGGGAGGCTCAAGGTCCCAGCCAGTGAGCCCAGTGTGCTTGAGTGTCTCTGGACTCG
CCCCTGAGCTTCCAGGTCTGTTTCATTTAGACTCCTGCTCGCCTCCGTGCAGTTGGGG
GAAAGCAAGAGACTTGCGCGCACGCACAGTCCTCTGGAGATCAGGTGGAAGGAGCCG
CTGGGTACCAAGGACTGTTCAGAGCCTCTTCCCATCTCGGGGAGAGCGAAGGGTGA
GGCTGGGCCCGGAGAGCAGTGTAAACGGCCTCCTCCGGCGGGATGGGAGCCATCGG
GCTCCTGTGGCTCCTGCCGCTGCTGCTTTCCACGGCAGCTGTGGGCTCCGGGATGGG
GACCGGCCAGCGCGCGGGCTCCCCAGCTGCGGGGCCGCCGCTGCAGCCCCGGGAG
CCACTCAGCTACTCGCGCCTGCAGAGGAAGAGTCTGGCAGTTGACTTCGTGGTGCCCT
CGCTCTTCCGTGTCTACGCCCGGGACCTACTGCTGCCACCATCCTCCTCGGAGCTGAA
GGCTGGCAGGCCCGAGGCCCGCGGCTCGCTAGCTCTGGACTGCGCCCCGCTGCTCA
GGTTGCTGGGGCCGGCGCCGGGGGTCTCCTGGACCGCCGGTTCACCAGCCCCGGCA
GAGGCCCGGACGCTGTCCAGGGTGCTGAAGGGCGGCTCCGTGCGCAAGCTCCGGCG
TGCCAAGCAGTTGGTGCTGGAGCTGGGCGAGGAGGCGATCTTGGAGGGTTGCGTCGG
GCCCCCCGGGGAGGCGGCTGTGGGGCTGCTCCAGTTCAATCTCAGCGAGCTGTTCAG
TTGGTGGATTCGCCAAGGCGAAGGGCGACTGAGGATCCGCCTGATGCCCGAGAAGAA
GGCGTCGGAAGTGGGCAGAGAGGGAAGGCTGTCCGCGGCAATTCGCGCCTCCCAGC
CCCGCCTTCTCTTCCAGATCTTCGGGACTGGTCATAGCTCCTTGGAATCACCAACAAAC
ATGCCTTCTCCTTCTCCTGATTATTTTACATGGAATCTCACCTGGATAATGAAAGACTCC
TTCCCTTTCCTGTCTCATCGCAGCCGATATGGTCTGGAGTGCAGCTTTGACTTCCCCTG
TGAGCTGGAGTATTCCCCTCCACTGCATGACCTCAGGAACCAGAGCTGGTCCTGGCGC
CGCATCCCCTCCGAGGAGGCCTCCCAGATGGACTTGCTGGATGGGCCTGGGGCAGAG
CGTTCTAAGGAGATGCCCAGAGGCTCCTTTCTCCTTCTCAACACCTCAGCTGACTCCAA
GCACACCATCCTGAGTCCGTGGATGAGGAGCAGCAGTGAGCACTGCACACTGGCCGT
CTCGGTGCACAGGCACCTGCAGCCCTCTGGAAGGTACATTGCCCAGCTGCTGCCCCA
CAACGAGGCTGCAAGAGAGATCCTCCTGATGCCCACTCCAGGGAAGCATGGTTGGACA
GTGCTCCAGGGAAGAATCGGGCGTCCAGACAACCCATTTCGAGTGGCCCTGGAATAC
ATCTCCAGTGGAAACCGCAGCTTGTCTGCAGTGGACTTCTTTGCCCTGAAGAACTGCA
GTGAAGGAACATCCCCAGGCTCCAAGATGGCCCTGCAGAGCTCCTTCACTTGTTGGAA
TGGGACAGTCCTCCAGCTTGGGCAGGCCTGTGACTTCCACCAGGACTGTGCCCAGGG
AGAAGATGAGAGCCAGATGTGCCGGAAACTGCCTGTGGGTTTTTACTGCAACTTTGAA
GATGGCTTCTGTGGCTGGACCCAAGGCACACTGTCACCCCACACTCCTC
AATGGCAGGTCAGGACCCTAAAGGATGCCCGGTTCCAGGACCACCAAGACCATGCTCT
ATTGCTCAGTACCACTGATGTCCCCGCTTCTGAAAGTGCTACAGTGACCAGTGCTACGT
TTCCTGCACCGATCAAGAGCTCTCCATGTGAGCTCCGAATGTCCTGGCTCATTCGTGGA
GTCTTGAGGGGAAACGTGTCCTTGGTGCTAGTGGAGAACAAAACCGGGAAGGAGCAA
GGCAGGATGGTCTGGCATGTCGCCGCCTATGAAGGCTTGAGCCTGTGGCAGTGGATG
GTGTTGCCTCTCCTCGATGTGTCTGACAGGTTCTGGCTGCAGATGGTCGCATGGTGGG
GACAAGGATCCAGAGCCATCGTGGCTTTTGACAATATCTCCATCAGCCTGGACTGCTAC
CTCACCATTAGCGGAGAGGACAAGATCCTGCAGAATACAGCACCCAAATCAAGAAACC
TGTTTGAGAGAAACCCAAACAAGGAGCTGAAACCCGGGGAAAATTCACCAAGACAGAC
CCCCATCTTTGACCCTACAGTTCATTGGCTGTTCACCACATGTGGGGCCAGCGGGCCC
CATGGCCCCACCCAGGCACAGTGCAACAACGCCTACCAGAACTCCAACCTGAGCGTG
GAGGTGGGGAGCGAGGGCCCCCTGAAAGGCATCCAGATCTGGAAGGTGCCAGCCACC
GACACCTACAGCATCTCGGGCTACGGAGCTGCTGGCGGGAAAGGCGGGAAGAACACC
ATGATGCGGTCCCACGGCGTGTCTGTGCTGGGCATCTTCAACCTGGAGAAGGATGACA
TGCTGTACATCCTGGTTGGGCAGCAGGGAGAGGACGCCTGCCCCAGTACAAACCAGTT
AATCCAGAAAGTCTGCATTGGAGAGAACAATGTGATAGAAGAAGAAATCCGTGTGAACA
GAAGCGTGCATGAGTGGGCAGGAGGCGGAGGAGGAGGGGGTGGAGCCACCTACGTA
TTTAAGATGAAGGATGGAGTGCCGGTGCCCCTGATCATTGCAGCCGGAGGTGGTGGCA
GGGCCTACGGGGCCAAGACAGACACGTTCCACCCAGAGAGACTGGAGAATAACTCCT
CGGTTCTAGGGCTAAACGGCAATTCCGGAGCCGCAGGTGGTGGAGGTGGCTGGAATG
ATAACACTTCCTTGCTCTGGGCCGGAAAATCTTTGCAGGAGGGTGCCACCGGAGGACA
TTCCTGCCCCCAGGCCATGAAGAAGTGGGGGTGGGAGACAAGAGGGGGTTTCGGAGG
GGGTGGAGGGGGGTGCTCCTCAGGTGGAGGAGGCGGAGGATATATAGGCGGCAATG
CAGCCTCAAACAATGACCCCGAAATGGATGGGGAAGATGGGGTTTCCTTCATCAGTCC
ACTGGGCATCCTGTACACCCCAGCTTTAAAAGTGATGGAAGGCCACGGGGAAGTGAAT
ATTAAGCATTATCTAAACTGCAGTCACTGTGAGGTAGACGAATGTCACATGGACCCTGA
AAGCCACAAGGTCATCTGCTTCTGTGACCACGGGACGGTGCTGGCTGAGGATGGCGT
CTCCTGCATTGTGTCACCCACCCCGGAGCCACACCTGCCACTCTCGCTGATCCTCTCT
GTGGTGACCTCTGCCCTCGTGGCCGCCCTGGTCCTGGCTTTCTCCGGCATCATGATTG
TGTACCGCCGGAAGCACCAGGAGCTGCAAGCCATGCAGATGGAGCTGCAGAGCCCTG
AGTACAAGCTGAGCAAGCTCCGCACCTCGACCATCATGACCGACTACAACCCCAACTA
CTGCTTTGCTGGCAAGACCTCCTCCATCAGTGACCTGAAGGAGGTGCCGCGGAAAAAC
ATCACCCTCATTCGGGGTCTGGGCCATGGCGCCTTTGGGGAGGTGTATGAAGGCCAG
GTGTCCGGAATGCCCAACGACCCAAGCCCCCTGCAAGTGGCTGTGAAGACGCTGCC
TGAAGTGTGCTCTGAACAGGACGAACTGGATTTCCTCATGGAAGCCCTGATCATCAGCA
AATTCAACCACCAGAACATTGTTCGCTGCATTGGGGTGAGCCTGCAATCCCTGCCCCG
GTTCATCCTGCTGGAGCTCATGGCGGGGGGAGACCTCAAGTCCTTCCTCCGAGAGACC
CGCCCTCGCCCGAGCCAGCCCTCCTCCCTGGCCATGCTGGACCTTCTGCACGTGGCT
CGGGACATTGCCTGTGGCTGTCAGTATTTGGAGGAAAACCACTTCATCCACCGAGACA
TTGCTGCCAGAAACTGCCTCTTGACCTGTCCAGGCCCTGGAAGAGTGGCCAAGATTGG
AGACTTCGGGATGGCCCGAGACATCTACAGGGCGAGCTACTATAGAAAGGGAGGCTGT
GCCATGCTGCCAGTTAAGTGGATGCCCCCAGAGGCCTTCATGGAAGGAATATTCACTT
CTAAAACAGACACATGGTCCTTTGGAGTGCTGCTATGGGAAATCTTTTCTCTTGGATATA
TGCCATACCCCAGCAAAAGCAACCAGGAAGTTCTGGAGTTTGTCACCAGTGGAGGCCG
GATGGACCCACCCAAGAACTGCCCTGGGCCTGTATACCGGATAATGACTCAGTGCTGG
CAACATCAGCCTGAAGACAGGCCCAACTTTGCCATCATTTTGGAGAGGATTGAATACTG
CACCCAGGACCCGGATGTAATCAACACCGCTTTGCCGATAGAATATGGTCCACTTGTG
GAAGAGGAAGAGAAAGTGCCTGTGAGGCCCAAGGACCCTGAGGGGGTTCCTCCTCTC
CTGGTCTCTCAACAGGCAAAACGGGAGGAGGAGCGCAGCCCAGCTGCCCCACCACCT
CTGCCTACCACCTCCTCTGGCAAGGCTGCAAAGAAACCCACAGCTGCAGAGATCTCTG
TTCGAGTCCCTAGAGGGCCGGCCGTGGAAGGGGGACACGTGAATATGGCATTCTCTCA
GTCCAACCCTCCTTCGGAGTTGCACAAGGTCCACGGATCCAGAAACAAGCCCACCAGC
TTGTGGAACCCAACGTACGGCTCCTGGTTTACAGAGAAACCCACCAAAAAGAATAATCC
TATAGCAAAGAAGGAGCCACACGACAGGGGTAACCTGGGGCTGGAGGGAAGCTGTAC
TGTCCCACCTAACGTTGCAACTGGGAGACTTCCGGGGGCCTCACTGCTCCTAGAGCCC
TCTTCGCTGACTGCCAATATGAAGGAGGTACCTCTGTTCAGGCTACGTCACTTCCCTTG
TGGGAATGTCAATTACGGCTACCAGCAACAGGGCTTGCCCTTAGAAGCCGCTACTGCC
CCTGGAGCTGGTCATTACGAGGATACCATTCTGAAAAGCAAGAATAGCATGAACCAGC
CTGGGCCCTGAGCTCGGTCGCACACTCACTTCTCTTCCTTGGGATCCCTAAGACCGTG
GAGGAGAGAGAGGCAATGGCTCCTTCACAAACCAGAGACCAAATGTCACGTTTTGTTTT
GTGCCAACCTATTTTGAAGTACCACCAAAAAAGCTGTATTTTGAAAATGCTTTAGAAAGG
TTTTGAGCATGGGTTCATCCTATTCTTTCGAAAGAAGAAAATATCATAAAAATGAGTGAT
AAATACAAGGCCCAGATGTGGTTGCATAAGGTTTTTATGCATGTTTGTTGTATACTTCCT
TATGCTTCTTTCAAATTGTGTGTGCTCTGCTTCAATGTAGTCAGAATTAGCTGCTTCTA
TGTTTCATAGTTGGGGTCATAGATGTTTCCTTGCCTTGTTGATGTGGACATGAGCCATTTG
AGGGGAGAGGGAACGGAAATAAAGGAGTTATTTGTAATGACTAAAA

A further example amino acid sequence of the ALK is the sequence deposited under NCBI Accession No. NP_001340694.1 ALK tyrosine kinase receptor isoform 2. This variant (2) represents use of an alternate promoter and therefore differs in the 5′ UTR and 5′ coding region, compared to variant 1. The promoter and 5′ terminal exon sequence is from an endogenous retroviral LTR. The resulting isoform (2, also known as ALKATI) is shorter and has a distinct N-terminus, compared to isoform 1.

The encoded protein is expressed in melanoma cells.

A further example amino acid sequence of the ALK is:

(SEQ ID NO: 3)
MQMELQSPEYKLSKLRTSTIMTDYNPNYCFAGKTSSISDLKEVPRKNITL
IRGLGHGAFGEVYEGQVSGMPNDPSPLQVAVKTLPEVCSEQDELDFLMEA
LIISKFNHQNIVRCIGVSLQSLPRFILLELMAGGDLKSFLRETRPRPSQP
SSLAMLDLLHVARDIACGCQYLEENHFIHRDIAARNCLLTCPGPGRVAKI
GDFGMARDIYRASYYRKGGCAMLPVKWMPPEAFMEGIFTSKTDTWSFGVL
LWEIFSLGYMPYPSKSNQEVLEFVTSGGRMDPPKNCPGPVYRIMTQCWQH
QPEDRPNFAIILERIEYCTQDPDVINTALPIEYGPLVEEEEKVPVRPKDP
EGVPPLLVSQQAKREEERSPAAPPPLPTTSSGKAAKKPTAAEISVRVPRG
PAVEGGHVNMAFSQSNPPSELHKVHGSRNKPTSLWNPTYGSWFTEKPTKK
NNPIAKKEPHDRGNLGLEGSCTVPPNVATGRLPGASLLLEPSSLTANMKE
VPLFRLRHFPCGNVNYGYQQQGLPLEAATAPGAGHYEDTILKSKNSMNQP
GP

A further example nucleotide sequence encoding the ALK is the sequence deposited under NCBI Accession No. NM_001353765.1

A further example nucleotide sequence encoding the ALK is:

(SEQ ID NO: 4)
CAGCCTTCCCTGGCTCCCTCCCCATTTCCTCTCATGGGCATTTCTTCTAA
TAAAATCTGCAGACCATATTGGGTCTAATCCCATCTCCAGTCTGCTTCTT
GGAGGAACCAGACTAACATGACTCTGCCCTATATAATACAAATAATTATT
TTCCATATATCTGATTTTTAGCTTTGCATTTACTTTAAATCATGCTTCAA
TTAAAGACACACCTTCTTTAATCATTTTATTAGTATTTCTAAGTATGATG
GAAAGGTTCAGAGCTCAGGGGAGGATATGGAGATCCAGGGAGGCTTCCTG
TAGGAAGTGGCCTGTGTAGTGCTTCAAGGGCCAGGCTGCCAGGCCATGTT
GCAGCTGACCACCCACCTGCAGTGTACCGCCGGAAGCACCAGGAGCTGCA
AGCCATGCAGATGGAGCTGCAGAGCCCTGAGTACAAGCTGAGCAAGCTCC
GCACCTCGACCATCATGACCGACTACAACCCCAACTACTGCTTTGCTGGC
AAGACCTCCTCCATCAGTGACCTGAAGGAGGTGCCGCGGAAAAACATCAC
CCTCATTCGGGGTCTGGGCCATGGCGCCTTTGGGGAGGTGTATGAAGGCC
AGGTGTCCGGAATGCCCAACGACCCAAGCCCCCTGCAAGTGGCTGTGAAG
ACGCTGCCTGAAGTGTGCTCTGAACAGGACGAACTGGATTTCCTCATGGA
AGCCCTGATCATCAGCAAATTCAACCACCAGAACATTGTTCGCTGCATTG
GGGTGAGCCTGCAATCCCTGCCCCGGTTCATCCTGCTGGAGCTCATGGCG
GGGGGAGACCTCAAGTCCTTCCTCCGAGAGACCCGCCCTCGCCCGAGCCA
GCCCTCCTCCCTGGCCATGCTGGACCTTCTGCACGTGGCTCGGGACATTG
CCTGTGGCTGTCAGTATTTGGAGGAAAACCACTTCATCCACCGAGACATT
GCTGCCAGAAACTGCCTCTTGACCTGTCCAGGCCCTGGAAGAGTGGCCAA
GATTGGAGACTTCGGGATGGCCCGAGACATCTACAGGGCGAGCTACTATA
GAAAGGGAGGCTGTGCCATGCTGCCAGTTAAGTGGATGCCCCCAGAGGCC
TTCATGGAAGGAATATTCACTTCTAAAACAGACACATGGTCCTTTGGAGT
GCTGCTATGGGAAATCTTTTCTCTTGGATATATGCCATACCCCAGCAAAA
GCAACCAGGAAGTTCTGGAGTTTGTCACCAGTGGAGGCCGGATGGACCCA
CCCAAGAACTGCCCTGGGCCTGTATACCGGATAATGACTCAGTGCTGGCA
ACATCAGCCTGAAGACAGGCCCAACTTTGCCATCATTTTGGAGAGGATTG
AATACTGCACCCAGGACCCGGATGTAATCAACACCGCTTTGCCGATAGAA
TATGGTCCACTTGTGGAAGAGGAAGAGAAAGTGCCTGTGAGGCCCAAGGA
CCCTGAGGGGGTTCCTCCTCTCCTGGTCTCTCAACAGGCAAAACGGGAGG
AGGAGCGCAGCCCAGCTGCCCCACCACCTCTGCCTACCACCTCCTCTGGC
AAGGCTGCAAAGAAACCCACAGCTGCAGAGATCTCTGTTCGAGTCCCTAG
AGGGCCGGCCGTGGAAGGGGGACACGTGAATATGGCATTCTCTCAGTCCA
ACCCTCCTTCGGAGTTGCACAAGGTCCACGGATCCAGAAACAAGCCCACC
AGCTTGTGGAACCCAACGTACGGCTCCTGGTTTACAGAGAAACCCACCAA
AAAGAATAATCCTATAGCAAAGAAGGAGCCACACGACAGGGGTAACCTGG
GGCTGGAGGGAAGCTGTACTGTCCCACCTAACGTTGCAACTGGGAGACTT
CCGGGGGCCTCACTGCTCCTAGAGCCCTCTTCGCTGACTGCCAATATGAA
GGAGGTACCTCTGTTCAGGCTACGTCACTTCCCTTGTGGGAATGTCAATT
ACGGCTACCAGCAACAGGGCTTGCCCTTAGAAGCCGCTACTGCCCCTGGA
GCTGGTCATTACGAGGATACCATTCTGAAAAGCAAGAATAGCATGAACCA
GCCTGGGCCCTGAGCTCGGTCGCACACTCACTTCTCTTCCTTGGGATCCC
TAAGACCGTGGAGGAGAGAGAGGCAATGGCTCCTTCACAAACCAGAGACC
AAATGTCACGTTTTGTTTTGTGCCAACCTATTTTGAAGTACCACCAAAAA
AGCTGTATTTTGAAAATGCTTTAGAAAGGTTTTGAGCATGGGTTCATCCT
ATTCTTTCGAAAGAAGAAAATATCATAAAAATGAGTGATAAATACAAGGC
CCAGATGTGGTTGCATAAGGTTTTTATGCATGTTTGTTGTATACTTCCTT
ATGCTTCTTTCAAATTGTGTGTGCTCTGCTTCAATGTAGTCAGAATTAGC
TGCTTCTATGTTTCATAGTTGGGGTCATAGATGTTTCCTTGCCTTGTTGA
TGTGGACATGAGCCATTTGAGGGGAGAGGGAACGGAAATAAAGGAGTTAT
TTGTAATGACTAA

In one embodiment, the ALK comprises an amino acid sequence that has at least 70%, 80%, 85%, 90%, 95%, 96%, 97%, 98%, 99% or 100% identity to SEQ ID NO: 1 or 3 preferably wherein the amino acid sequence substantially retains the natural function of the protein represented by SEQ ID NO: 1 or 3.

In one embodiment, the ALK-encoding nucleotide sequence comprises a nucleotide sequence that has at least 70%, 80%, 85%, 90%, 95%, 96%, 97%, 98%, 99% or 100% identity to SEQ ID NO: 2 or 4, preferably wherein the protein encoded by the nucleotide sequence substantially retains the natural function of the protein represented by SEQ ID NO: 1 or 3.

In one embodiment, the ALK-encoding nucleotide sequence comprises a nucleotide sequence that encodes an amino acid sequence that has at least 70%, 80%, 85%, 90%, 95%, 96%, 97%, 98%, 99% or 100% identity to SEQ ID NO: 1 or 3 preferably wherein the amino acid sequence substantially retains the natural function of the protein represented by SEQ ID NO: 1 or 3.

### Weight Loss and Weight Maintenance

The term “weight loss” as used herein may refer to a reduction in parameters such as weight (e.g. in kilograms), body mass index (kg/m2), waist-hip ratio (e.g. in centimetres), fat mass (e.g. in kilograms), hip circumference (e.g. in centimetres) or waist circumference (e.g. in centimetres).

Weight loss may be calculated by subtracting the value of one or more of the aforementioned parameters at the end of an intervention (e.g. a diet and/or exercise regimen) from the value of the parameter at the onset of the intervention.

The degree of weight loss may be expressed as a percent change of one of the aforementioned weight phenotype parameters (e.g. a percent change in a subject's body weight (e.g. in kilograms) or body mass index (kg/m2)). For example, a subject may lose at least 10% of their initial body weight, at least 8% of their initial body weight, or at least 5% of their initial body weight. By way of example only, a subject may lose between 5 and 10% of their initial body weight.

In one embodiment, a degree of weight loss of at least 10% of initial body weight results in a considerable decrease in the risk of obesity-related co-morbidities.

The term “weight maintenance” as used herein may refer to the maintenance in parameters such as weight (e.g. in kilograms), body mass index (kg/m2), waist-hip ratio (e.g. in centimetres) fat mass (e.g. in kilograms), hip circumference (e.g. in centimetres) or waist circumference (e.g. in centimetres). Weight maintenance may refer to, for example, maintaining weight lost following an intervention (e.g. a diet and/or exercise regimen).

The degree of weight maintenance may be calculated by determining the change in one or more of the afore-mentioned parameters over a period of time. The period of time may be, for example, at least 5, 10, 15, 20, 25, 30, 35, 40, 45 or 50 weeks.

Weight maintenance supported by the agents of the invention may result in, for example, a change (e.g. gain) of less than 10%, 9%, 8%, 7%, 6%, 5%, 4%, 3%, 2% or 1% in one or more of the afore-mentioned parameters over a period of time.

The degree of weight maintenance may be expressed as the weight regained during a period following attainment of weight loss, for example as a percentage of the weight lost during attainment of weight loss.

Weight maintenance supported by the agents of the invention may result through suppression of a subject's appetite following administration of the agent. The subject may therefore have a reduced appetite compared to the appetite in the absence of the agent of the invention.

Weight maintenance supported by the agents of the invention may result through control of a subject's appetite following administration of the agent. The subject may therefore maintain control over their appetite and therefore maintain their weight, for example following a period of weight loss intervention.

In particular, the agents of the invention may support weight maintenance through appetite suppression or control during and/or following a period of weight loss intervention (e.g. a diet or exercise regime).

In one aspect, the invention provides the non-therapeutic use of an agent of the invention to maintain a healthy body composition, for example after a period of weight loss.

### Obesity

The term “overweight” as used herein is defined for an adult human as having a body mass index (BMI) between 25 and 30.

The term “body mass index” as used herein means the ratio of weight in kg divided by the height in metres, squared.

The term “obesity” as used herein refers to a condition in which the natural energy reserve, stored in the fatty tissue of animals, in particular humans and other mammals, is increased to a point where it is associated with certain health conditions or increased mortality. The term “obese” as used herein is defined for an adult human as having a BMI greater than 30.

The term “normal weight” as used herein is defined for an adult human as having a BMI of 18.5 to 25, whereas the term “underweight” as used herein may be defined as a BMI of less than 18.5.

Obesity is a chronic metabolic disorder that has reached epidemic proportions in many areas of the world and is the major risk factor for serious co-morbidities such as type 2 diabetes mellitus, cardiovascular disease, dyslipidemia and certain types of cancer (World Health Organ. Tech. Rep. Ser. (2000) 894: i-xii, 1-253).

The term “obesity-related disorder” as used herein refers to any condition which an obese individual is at an increased risk of developing. Obesity-related disorders include diabetes (e.g. type 2 diabetes), stroke, high cholesterol, cardiovascular disease, insulin resistance, coronary heart disease, metabolic syndrome, hypertension and fatty liver.

### Methods of Screening

The invention provides agents that are capable of decreasing the activity of ALK, and additionally provides methods for identifying such agents.

The agents of the invention may be identified by methods that provide either qualitative or quantitative results. Furthermore, such methods may be used to characterise as well as identify agents of the invention.

The candidate agents may be any agents of potential interest, for example peptides, polypeptides (e.g. antibodies), nucleic acids or small molecules. Preferably, the candidate agents are compounds or mixtures of potential therapeutic interest. Preferably, the candidate agents are of low toxicity for mammals, in particular humans. In some embodiments, the candidate agents may comprise nutritional agents and/or food ingredients, including naturally-occurring compounds or mixtures of compounds such as plant or animal extracts.

The candidate agents may form part of a library of agents, for example a library produced by combinatorial chemistry or a phage display library. In one embodiment, the candidate agents form part of a library of plant bioactive molecules.

### ALK Activity

The ability of a candidate agent to reduce the activity of a protein, for example an enzyme, may be expressed in terms of an IC50 value. The IC50 is the concentration of an agent that is required to give rise to a 50% reduction in the activity of the protein (e.g. a 50% reduction in enzymatic activity). The calculation of IC50 values is well known in the art.

Preferably, the agents of the invention have an IC50 value for inhibition of ALK of less than 100 M, more preferably less than 10 UM, for example less than 1 μM, less than 100 nM or less than 10 nM.

Techniques for measuring ALK activity may be applied to ALK that has been isolated from a cell. The ALK may have been expressed using recombinant techniques. Preferably, the ALK has been purified.

ALK binding

The invention also provides methods of identifying agents which are capable of binding to ALK and, alternatively or additionally, characterising such binding. For example, the method may allow measurement of absolute or relative binding affinity, and/or enthalpy and entropy of binding. Binding affinity may be expressed in terms of the equilibrium dissociation (Ka) or association (Ka) constant.

A number of assay techniques are known in the art for identifying binding between a candidate agent and a protein. The assay technique employed is preferably one which is amenable to automation and/or high throughput screening of candidate agents. The assay may be performed on a disposable solid support such as a microtitre plate, microbead, resin or similar.

For example, target ALK may be immobilised on a solid support, for example a microbead, resin, microtitre plate or array. Candidate agents may then be contacted with the immobilised target protein. Optionally, a wash procedure may be applied to remove weakly or non-specifically binding agents.

Any agents binding to the target protein may then be detected and identified. To facilitate the detection of bound agents, the candidate agents may be labelled with a readily detectable marker. The marker may comprise, for example, a radio label, an enzyme label, an antibody label, a fluorescent label, a particulate (e.g. latex or gold) label or similar.

Alternatively, the above procedure may be reversed and the candidate agents may be immobilised and the target ALK may be contacted with said immobilised agents. Optionally, a wash procedure may be applied to remove weakly or non-specifically bound target protein. Any agents to which ALK binds may then be detected and identified. To facilitate the detection of binding, the ALK may be labelled with a readily detectable marker as described above.

In addition to the assays described above, other suitable assay techniques are known in the art.

Examples of such techniques include radioassays, fluorescence assays, ELISA, fluorescence polarisation, fluorescence anisotropy, isothermal titration calorimetry (ITC), surface plasmon resonance (SPR) and the like. These assays may be applied to identify agents which bind to ALK. Indeed, platforms for the automation of many of these techniques are widely known in the art to facilitate high-throughput screening.

More than one assay technique may be used to provide a detailed understanding of a candidate agent's binding to ALK. For example, assays which provide qualitative binding information may be used as a first step in the method, followed by further assays using different techniques to provide quantitative binding data and/or data on the effect on activity of the target protein.

The assay techniques described above may be adapted to perform competition binding studies. For example, these techniques are equally suitable to analyse the binding of a protein to substrate or cofactor in the presence of a candidate agent. It will therefore be possible to use the above techniques to screen and identify agents that modulate the binding between a protein and its substrate or cofactor, thus having an effect on the protein's activity.

Preferably, the agents of the invention will bind with high affinity. For example, the agents of the invention will bind to ALK with a K_{d} of less than 100 μM, more preferably less than 10 M, for example less than 1 μM, less than 100 nM or less than 10 nM.

Binding affinity may be measured using standard techniques known in the art, e.g. surface plasmon resonance, ELISA and so on (for instance as described above), and may be quantified in terms of either dissociation (Ka) or association (Ka) constants.

Bioinformatics-based approaches, such as in silico structure-guided screening, may also be used to identify agents of the invention.

ALK levels

The invention provides agents for decreasing ALK levels. Levels of ALK may be equated with levels of expression of the protein in a cell or organism. Protein levels may be analysed directly or indirectly, for example by analysis of levels of mRNA encoding the protein.

Methods for analysing the expression of ALK may be employed in the invention to screen the effect of a candidate agent on the protein's levels.

A number of techniques are known in the art for determining the expression level of a protein. These techniques may be applied to test the effect of candidate agents on the expression level of ALK. The technique employed is preferably one which is amenable to automation and/or high throughput screening of candidate agents.

For example, screens may be carried out using cells harbouring polynucleotides encoding ALK operably linked to a reporter moiety. The reporter moiety may be operably linked to endogenous ALK-encoding genes. Alternatively, exogenous copies of ALK operably linked to a reporter moiety may be inserted into a cell. In this embodiment, the cell may be engineered to be deficient for natural ALK expression. Suitable reporter moieties include fluorescent labels, for example fluorescent proteins such as green, yellow, cherry, cyan or orange fluorescent proteins.

The term “operably linked” as used herein means the components described are in a relationship permitting them to function in their intended manner.

Such cells may be contacted with candidate agents and the level of expression of ALK may be monitored by analysing the level of reporter moiety expression in the cell. Fluorescent reporter moieties may be analysed by a number of techniques known in the art, for example flow cytometry, fluorescence activated cell sorting (FAGS) and fluorescence microscopy. Expression levels of ALK may be compared before and after contact with the candidate agent. Alternatively, expression levels of ALK may be compared between cells contacted with a candidate agent and control cells.

Other methods may be used for analysing the expression of proteins, for example ALK. Protein expression may be analysed directly. For example, expression may be quantitatively analysed using methods such as SDS-PAGE analysis with visualisation by Coomassie or silver staining. Alternatively, expression may be quantitatively analysed using Western blotting or enzyme-linked immunosorbent assays (ELISA) with antibody probes which bind the protein product. ALK labelled with reporter moieties, as described above, may also be used in these methods. Alternatively, protein expression may be analysed indirectly, for example by studying the amount of mRNA corresponding to the protein that is transcribed in a cell. This can be achieved using methods such as quantitative reverse transcription PCR and Northern blotting.

Similar techniques may also be used for the analysis of leptin protein expression. Agents The invention provides agents that are capable of decreasing the activity of ALK, and additionally provides methods for identifying such agents.

The agents of the invention may be, for example, peptides, polypeptides (e.g. antibodies), nucleic acids (e.g. siRNAs, shRNAs, miRNAs and antisense RNAs) or small molecules. Preferably, the agents are of low toxicity for mammals, in particular humans. In some embodiments, the agents may comprise nutritional agents and/or food ingredients, including naturally-occurring compounds or mixtures of compounds such as plant or animal extracts.

Example agents that decrease or otherwise affect the activity of ALK include the agents recited in Table 1.

TABLE 1
Agents that decrease or otherwise affect the activity of ALK.

| | | | | Reference/ |
|---|---|---|---|---|
| Chemical Name | Chemical ID | CAS RN | Interaction Actions | patent number |
| Acetylcysteine | D000111 | 616-91-1 | affects activity | http://ctdbase.org/ |
| alectinib | | | inhibit | WO-2017053657-A1 |
| Brigatinib | | 1197953-54-0 | inhibit | http://ctdbase.org/ |
| ceritinib | | 1032900-25-6 | inhibit | WO-2017175111-A1 |
| crizotinib | | 877399-52-5 | inhibit | U.S.-20170007561-A1, |
| | | | | U.S.-9,446,039-B2, |
| | | | | WO-2012075318-A2 |
| emodin | | 518-82-1 | inhibit | U.S.-20150202204-A1 |
| everolimus | | 159351-69-6 | inhibit | Medline |
| foretinib | | 849217-64-7 | inhibit | WO-2014134096-A1 |
| heparin | | 9005-49-6 | terminate | U.S.-20170100428-A1 |
| herbimycin | | 70563-58-5 | inhibit | U.S.-8,822,500-B2 |
| hydrocortisone | | 50-23-7 | suppress | Medline |
| lapatinib | | 231277-92-2 | inhibit | WO-2017037220-A1 |
| Masoprocol | D009637 | | decreases activity | http://ctdbase.org/ |
| NVP-TAE684 | C516714 | | decreases activity | http://ctdbase.org/ |
| PF-04254644 | C551178 | | affects activity | http://ctdbase.org/ |
| pyrimidine | | | inhibit | WO-2016192132-A1 |
| Simvastatin | D019821 | 79902-63-9 | affects cotreatment, | http://ctdbase.org/ |
| | | | decreases expression | |
| sucrose octasulfate | | | inhibit | U.S.-20170100428-A1 |
| tanespimycin | C112765 | | decreases activity | http://ctdbase.org/ |
| tivozanib | | | inhibit | WO-2017037220-A1 |
| Tretinoin | D014212 | 302-79-4 | decreases expression | http://ctdbase.org/ |
| vibramycin | | 564-25-0 | inhibit | U.S.-7,732,592-B2 |

The agents for use according to the invention may be, for example, present as salts or esters, in particular pharmaceutically acceptable salts or esters.

siRNAs, shRNAs, miRNAs and antisense DNAs/RNAs

Expression of ALK may be modulated using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11: 429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA>30 bp has been found to activate the interferon response leading to shut-down of protein synthesis and non-specific mRNA degradation (Stark et al. (1998) Ann. Rev. Biochem. 67: 227-64). However, this response can be bypassed by using 21 nt siRNA duplexes (Elbashir et al. (2001) EMBO J. 20: 6877-88; Hutvagner et al. (2001) Science 293: 834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) non-coding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an −70 nt precursor, which is post-transcriptionally processed into a mature −21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art. Furthermore, methods for specifically modulating (e.g. reducing) expression of a protein in a certain cell type within an organism, for example through the use of tissue-specific promoters are well known in the art.

### Antibodies

The term “antibody” as used herein refers to complete antibodies or antibody fragments capable of binding to a selected target, and includes Fv, ScFv, F(ab′) and F(ab′)2, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

Methods for the production of antibodies are known by the skilled person. Alternatively, antibodies may be derived from commercial sources.

If polyclonal antibodies are desired, a selected mammal (e.g. mouse, rabbit, goat or horse) may be immunised. Serum from the immunised animal may be collected and treated according to known procedures. If the serum contains polyclonal antibodies to other antigens, the polyclonal antibodies may be purified by immunoaffinity chromatography. Techniques for producing and processing polyclonal antisera are known in the art.

Monoclonal antibodies directed against antigens (e.g. proteins) used in the invention can also be readily produced by the skilled person. The general methodology for making monoclonal antibodies by hybridomas is well known. Immortal antibody-producing cell lines can be created by cell fusion and also by other techniques such as direct transformation of B-lymphocytes with oncogenic DNA or transfection with Epstein-Barr virus. Panels of monoclonal antibodies produced against antigens can be screened for various properties, for example for isotype and epitope affinity.

An alternative technique involves screening phage display libraries where, for example, the phage express scFv fragments on the surface of their coat with a large variety of complementarity determining regions (CDRs). This technique is well known in the art.

Antibodies, both monoclonal and polyclonal, which are directed against antigens, are particularly useful in diagnosis, and those which are neutralising are useful in passive immunotherapy. Monoclonal antibodies in particular may be used to raise anti-idiotype antibodies. Anti-idiotype antibodies are immunoglobulins which carry an “internal image” of the antigen of the infectious agent against which protection is desired.

Techniques for raising anti-idiotype antibodies are known in the art. These anti-idiotype antibodies may also be useful for treatment, as well as for an elucidation of the immunogenic regions of antigens.

Introduction of Polypeptides and Polynucleotides into Cells

An agent for use in the invention may be, for example, a polypeptide or a polynucleotide. Polynucleotides and polypeptides may also need to be introduced into cells as part of the methods or screening assays of the invention.

Where the invention makes use of a polypeptide, the polypeptides may be administered directly to a cell (e.g. the polypeptide itself may be administered), or the polypeptides may be administered by introducing polynucleotides encoding the polypeptide into cells under conditions that allow for expression of the polypeptide in a cell of interest. Polynucleotides may be introduced into cells using vectors.

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred to a target cell. The vector may serve the purpose of maintaining the heterologous nucleic acid (e.g. DNA or RNA) within the cell, facilitating the replication of the vector comprising a segment of nucleic acid or facilitating the expression of the protein encoded by a segment of nucleic acid. Vectors may be non-viral or viral. Examples of vectors used in recombinant nucleic acid techniques include, but are not limited to, plasmids, chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid or virus vectors and may include a promoter for the expression of a polynucleotide and optionally a regulator of the promoter.

Vectors comprising polynucleotides used in the invention may be introduced into cells using a variety of techniques known in the art, such as transduction and transfection. Several techniques suitable for this purpose are known in the art, for example infection with recombinant viral vectors, such as retroviral, lentiviral, adenoviral, adeno-associated viral, baculoviral and herpes simplex viral vectors; direct injection of nucleic acids and biolistic transformation. Non-viral delivery systems include, but are not limited to, DNA transfection methods. Transfection includes a process using a non-viral vector to deliver a gene to a target cell.

Transfer of the polypeptide or polynucleotide may be performed by any of the methods known in the art which may physically or chemically permeabilise the cell membrane. Cell-penetrating peptides may also be used to transfer a polypeptide into a cell.

In addition, the invention may employ gene targeting protocols, for example the delivery of DNA-modifying agents.

The vector may be an expression vector. Expression vectors as described herein comprise regions of nucleic acid containing sequences capable of being transcribed. Thus, sequences encoding mRNA, tRNA and rRNA are included within this definition.

Expression vectors preferably comprise a polynucleotide for use in the invention operably linked to a control sequence that is capable of providing for the expression of the coding sequence by the host cell. A regulatory sequence “operably linked” to a coding sequence is ligated in such a way that expression of the coding sequence is achieved under conditions compatible with the control sequence. The control sequence may be modified, for example by the addition of further transcriptional regulatory elements to make the level of transcription directed by the control sequence more responsive to transcriptional modulators.

### Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides, such as DNA polynucleotides, may be produced recombinantly, synthetically or by any means available to the skilled person. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA, for example mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Proteins

The term “protein” as used herein includes single chain polypeptide molecules as well as multiple-polypeptide complexes where individual constituent polypeptides are linked by covalent or non-covalent means. The terms “polypeptide” and “peptide” as used herein refer to a polymer in which the monomers are amino acids and are joined together through peptide or disulfide bonds.

### Variants, Derivatives, Analogues, Homologues and Fragments

In addition to the specific proteins and nucleotides mentioned herein, the invention also encompasses variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | GAP |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”

In the present context, a homologous sequence is taken to include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein.

These methods are only illustrative of the numerous standard techniques known in the art for manipulation of DNA sequences and other known techniques may also be used.

### Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding RNAs are known to be rare in the particular cell type. Thus, an additional degree of translational control is available. Codon usage tables are known in the art for mammalian cells, as well as for a variety of other organisms.

### Method of Treatment

All references herein to treatment include curative, palliative and prophylactic treatment. The treatment of mammals, particularly humans, is preferred. Both human and veterinary treatments are within the scope of the invention.

### Administration

Although the agents for use in the invention can be administered alone, they will generally be administered in admixture with a pharmaceutical carrier, excipient or diluent, particularly for human therapy.

In some embodiments, the agent is a nutritional agent, food additive or food ingredient, and may thus be formulated in a suitable food composition. Thus, the agent may be administered, for example, in the form of a food product, drink, food supplement, nutraceutical, nutritional formula or pet food product.

### Dosage

The skilled person can readily determine an appropriate dose of an agent of the invention to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats.

The non-human animal may be a companion animal.

Preferably, the subject is a human.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1: Link Between ALK Genetic Variants and a Thin Phenotype

Population selection. This study relates to a genetic association study performed on Constitutional Thin (CT) individuals using data from the EGCUT biobank. Estonia, through the Estonian Genomic Center of the University of Tartu (EGCUT), has set-up a biobank based on the general population with biological samples (DNA, RNA and plasma) and clinical data from 60,000 individuals. The thin phenotype was defined as those people with the lowest 6th percentile BMI adjusted for age and sex, and strict exclusion criteria included pregnancy, postmenopausal or menstrual abnormalities, subjects who are vegetarian or have known intolerances (allergies) to food, subjects with eating disorders according to DSMIV, consumption of more than 10 glasses of wine per week, severe chronic disease (e.g. diabetes), excessive physical activity (more than 3 training sessions per week), more than 10 cigarettes per week, depression or psychiatric condition (anti-depressant treatment), past surgery known to influence weight (especially bariatric surgery), subjects under known treatment with beta-blockers, anti-hypertensives, lipid lowering drugs or corticoids for a long duration, cancer patients. Analysis of their database showed that 800 CT individuals were identified, as well as 3000 matched control individuals. This resource allows a powerful and controlled genetic study of the CT phenotype. It is the first study to test the genetic component of the CT phenotype on a large scale.

Genotyping. Genotype data were generated using HumanCorePsy array (www.11lumina.com). Genotypes were called with the GenomeStudio Software (Illumina). Rare variant genotype calling was performed using zCall (Goldstein et al. Bioinformatics (2012)). Genotype quality control removed any subject with
SNP call rate<98%
Any gender discrepancies between known gender and inferred gender from genotype data
Genotype heterozygosity>3 standard deviations
Population outlier (as detected with MDS analyses)

### Quality Control Also Excluded any Marker if

Call rate<95%
Deviation from Hardy-Weinberg equilibrium (p<1e-4)
Rare variant with Minor Allele Frequency (MAF)<1%
A/T or C/G genotypes (prior imputation)

Genotype imputation was then performed using SHAPEIT (Delaneau, 0., Marchini, J. & Zagury, J.-F. Nat Meth 9, 179-181 (2012)) and IMPUTE2 (Howie, B. N., Donnelly, P. & Marchini, J. PLOS Genet. 5, e1000529 (2009)) based reference panels from the 1000 Genome project (Abecasis, G. R. et al. Nature 491, 56-65 (2012)) (1000 Genomes integrated haplotypes, Dec. 2013 release). Post-imputation quality control removed markers with INFO score <0.8 or MAF <1%.

In total, the genotype data consisted in 281K genotyped and 8.3M imputed markers.

Statistical Analysis. Statistical analysis was performed with SNPTEST (Marchini, J. et al. Nature Genetics (2007)), using the method ‘expected’. Such logistic regression compared genotypes between CT and controls. Covariates included gender, age and the four first components from a Principal Component Analysis on genotype data (to account for possible population stratification). Upon analysis, no abnormal p-value inflation were seen on QQ plots.

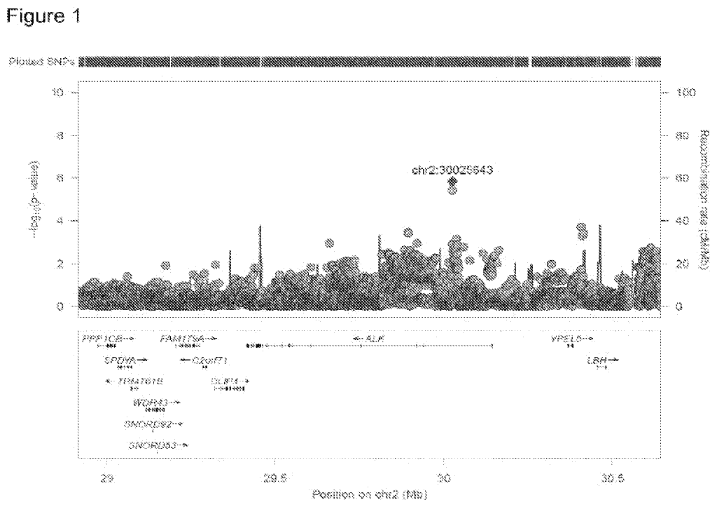

Results. Analyses identified rs568057364 (also referred to as chr2:30025643) as top variant 5 associated with the CT phenotype (p=1.44e-6) (Table 2). This variant is an indel, located in the intronic region of the ALK gene (FIG. 1). The second top variant is rs202021741 (chr2:30025449, with p=3.8e-6). Both hits are annotated as regulatory variants.

TABLE 2
Top associations with the CT phenotype, in the ALK gene.

| CHR | POS | MARKER | EA | NEA | EAF | STRAND | OR | OR_95L | OR_95U | P |
|---|---|---|---|---|---|---|---|---|---|---|
| 2 | 30025449 | rs202021741:30025449:GGAAGA:G | G | GGAAGA | 0.4654 | + | 1.30 | 1.166 | 1.463 | 3.80E−06 |
| 2 | 30025643 | rs568057364:2:30025643:C:CT | CT | C | 0.4692 | + | 1.3358 | 1.183 | 1.488 | 1.44E−06 |

Abbreviations: CHR: chromosome name; POS: position on chromosome (basepairs); EA and NEA: effective and non-effective alleles; OR, OR 95L and OR 95U: Odds ratios with 95% confidence intervals; P: association p-value.

### Example 2: In Vivo Function of ALK

15 Fly strains. Fly stocks were maintained on standard diet with agar, sugar and yeast and were raised in 25° ° C. incubator at a 12/12 dark and night cycle. Actin-Gal4 was from Bloomington and w1118 and UAS-AlkIR (GD 11446) were from the VDRC.

Triglyceride assay. 5 times 10 (4-7 days old) male flies were weighted and homogenised in 200 μl dH₂O on ice, then sonicated for 10 s using a probe sonicator on ice. After sonication, 800 μl ice-cold dH₂O was added and mixed thoroughly. 50 μl of the mixture was used to determine the triglycerides using Roche triglycerides kits (11730711216) under manufacture's instructions. Body weight was measured by analytic balance. Triglycerides were normalized to body weight.

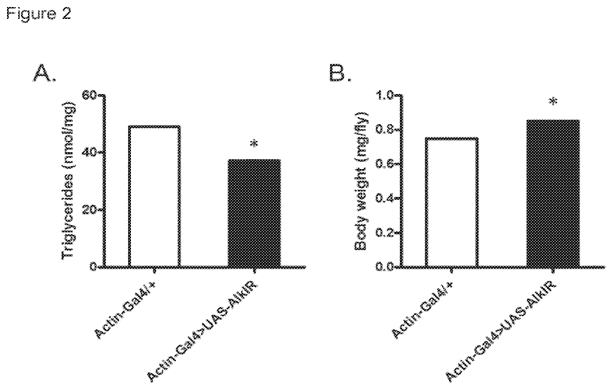

Results. To investigate ALK gene function in vivo we used transgenic RNAi in the fruit fly Drosophila melanogaster. ALK showed high homology with the Alk gene in fly. Using whole body (Actin-Gal4) driver, Alk mutant animals (Actin-Gal4>UAS-AlkIR) were viable with no overt developmental phenotype. Importantly, Alk knockdown animals exhibited marked (24%) decrease in triglyceride accumulation (Z-score=−3.27) (FIG. 2A) compared to controls (Actin-Gal4/+), while body weight was increased (Z-score=3.02) (13%) (FIG. 2B). Thus, targeting Alk in vivo promotes a lean phenotype in the fly.

All publications mentioned in the above specification are herein incorporated by reference. Various modifications and variations of the disclosed agents, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1-17. (canceled)

18. A method of preparing a food composition, the method comprising:
(a) contacting a preparation comprising a polypeptide or polynucleotide of Anaplastic Lymphoma Receptor Tyrosine Kinase (ALK) with a candidate agent;
(b) detecting whether the candidate agent decreases activity of the polypeptide or polynucleotide of ALK to thereby identify an agent capable of decreasing activity of ALK;
(c) using the agent capable of decreasing the activity of ALK as a nutritional agent, food additive or food ingredient, in the food composition selected from the group consisting of a food product, a drink, a food supplement, a nutraceutical, a nutritional formula and a pet food product; and
(d) providing the food composition comprising the agent capable of decreasing the activity of ALK to an individual having obesity, at risk of obesity, and/or in need of weight maintenance.

19. The method according to claim 18, wherein the food composition comprising the agent is provided to the individual during or after a weight loss intervention.

20. The method according to claim 18, wherein the agent decreases a level of ALK in the individual.

21. The method according to claim 18, wherein the agent is selected from the group consisting of Acetylcysteine, alectinib, Brigatinib, ceritinib, crizotinib, emodin, everolimus, foretinib, heparin, herbimycin, hydrocortisone, lapatinib, Masoprocol, NVP-TAE684, PF-04254644, pyrimidine, Simvastatin, sucrose octasulfate, tanespimycin, tivozanib, Tretinoin, and vibramycin.

22. A method of identifying an agent capable of supporting weight maintenance and/or treating or preventing obesity in a subject, the method comprising:
(a) contacting a preparation comprising a ALK polypeptide or polynucleotide with a candidate agent; and
(b) detecting whether the candidate agent affects the activity of the ALK polypeptide or polynucleotide.

23. A method of identifying an agent that decreases the activity of ALK, the method comprising:
(a) contacting a preparation comprising a ALK polypeptide or polynucleotide with a candidate agent; and
(b) detecting whether the candidate agent affects the activity of the ALK polypeptide or polynucleotide.

24. The method of claim 22, wherein the preparation comprising the ALK polypeptide or polynucleotide comprises a cell comprising the ALK polypeptide or polynucleotide.

25. The method of claim 24, wherein the cell is an adipocyte.

26. The method of claim 24, wherein the cell is a brain cell.

27. The method of claim 22, wherein the method identifies an agent that decreases the expression of ALK.

28. The method of claim 22, wherein the candidate agent is a natural product.

29. The method of claim 23, wherein the preparation comprising the ALK polypeptide or polynucleotide comprises a cell comprising the ALK polypeptide or polynucleotide.

30. The method of claim 29, wherein the cell is an adipocyte.

31. The method of claim 29, wherein the cell is a brain cell.

32. The method of claim 23, wherein the method identifies an agent that decreases the expression of ALK.

33. The method of claim 23, wherein the candidate agent is a natural product.

34. The method of claim 18, wherein the food composition comprising the agent is provided to the individual after a period of weight loss, in an amount effective for resistance to weight gain.

35. The method according to claim 19, wherein the food composition comprising the agent is provided to the individual during a weight loss intervention.

36. A method of preparing a food composition, the method comprising:
(a) contacting a preparation comprising a polypeptide or polynucleotide of Anaplastic Lymphoma Receptor Tyrosine Kinase (ALK) with a candidate agent;
(b) detecting whether the candidate agent decreases activity of the polypeptide or polynucleotide of ALK to thereby identify an agent capable of decreasing activity of ALK; and
(c) using the agent capable of decreasing the activity of ALK as a nutritional agent, food additive or food ingredient, in the food composition selected from the group consisting of a food product, a drink, a food supplement, a nutraceutical, a nutritional formula and a pet food product.


SEQUENCE LISTING (4 sequences)

SEQ ID NO: 1 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 2 - DNA, 6267 bp, Homo sapiens
  source 1..6267: mol_type = genomic DNA
agctgcaagtggcgggcgcccaggcagatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 3 - protein, 552 aa, Homo sapiens
MQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 4 - DNA, 2513 bp, Homo sapiens
  source 1..2513: mol_type = genomic DNA
cagccttccctggctccctccccatttcctctcatgggcatttcttctaataaaatctgcagaccatattgggtctaatcccatctccagtctgcttcttggaggaaccagactaacatgactctgccctatataatacaaataattattttccatatatctgatttttagctttgcatttactttaaatcatgcttcaattaaagacacaccttctttaatcattttattagtatttctaagtatgatggaaaggttcagagctcaggggaggatatggagatccagggaggcttcctgtaggaagtggcctgtgtagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa